(19) United States
(12) Patent Application Publication
(10) Pub. No.: US 2010/0068716 A1
(43) Pub. Date: Mar. 18, 2010

(54) DISPOSABLE ARTICLES FOR ANALYSIS AND DIAGNOSTICS FOR A LABORATORY

(76) Inventor: Wolfgang Weber, Berlin (DE)

Correspondence Address: DICKSTEIN SHAPIRO LLP, 1825 EYE STREET NW, Washington, DC, 20006-5403, US

(21) Appl. No.: 12/516,738

(22) PCT Filed: Dec. 3, 2007
(86) PCT No.: PCT/EP07/63192

§ 371 Date: Oct. 22, 2009

(30) Foreign Application Priority Data
Dec. 1, 2006 (DE) 102006056790.0

(51) Int. Cl. C12Q 1/68; C12M 1/00; C12P 19/34

(52) U.S. Cl. 435/6; 435/289.1; 435/91.2

(57) ABSTRACT

The invention relates to disposable articles for a laboratory and in particular to prepared reaction vessels for conducting the polymerase chain reaction for analytical and diagnostic purposes. The invention further relates to a method for the stable storage and drying of minute amounts of oligonucleotides and reference nucleic acids in reaction vessels. The oligonucleotides and the nucleic acids are dried on the wall of the reaction vessel in the presence of 1 to 5 mM trehalose, without any further components.

### FIELD OF THE INVENTION

The invention relates to disposable laboratory articles and in particular to reaction vessels for carrying out polymerase chain reactions for analytical and diagnostic purposes.

### STATE OF THE ART

The Polymerase Chain Reaction (PCR) enables an exponential amplification of nucleic acid molecules in vitro. PCR is used for the diagnosis of hereditary and infectious diseases, for the determination of genetic fingerprints, cloning of genes, determination of paternity and many other applications. It is used in food chemistry for the determination of the type and amounts of proportions in a formulation, such as hazelnut, peanut, soy-bean, fish, wheat, grains, animal species and optionally genetically modified organisms (GMOs), the origin of the constituents, and of course for the determination of the presence of pathogenic germs, such as salmonella, listeria and so on. Despite the many applications, the basic procedure of PCR in principle always remains the same. The analyses essentially only differ in the work-up of the samples and the type of DNA or RNA which is to be amplified, or the starting oligonucleotides (primers), the sequences of which have to be complementary to the start or the end of a DNA sequence to be amplified. The primers are bound by annealing to a complementary nucleotide strand in the sample, if present, and the synthetically produced new double-strands then contain further starting points for the synthesis of more DNA-strands. Sensitivity and specificity of the reaction are given by the length and sequence of the primer and the outstanding fidelity of the enzymatic DNA-synthesis by DNA-polymerases or reverse transcriptase. In general, the optimal length of the primer is between 15 and 40 nucleotides with a melting temperature between 55 and 70° C. Apart from the specific primers, the reaction mixture for the PCR always contains the same deoxynucleotide triphosphates (dNTPs), an aqueous buffer solution, DNA-polymerase or reverse transcriptase and, in addition, DNA or RNA of the sample to be analysed. After PCR, the synthesised DNA product is normally analysed for its length and, if applicable, verified by enzymatic restriction. In particular cases, a sequencing of the DNA may take place.

Even though PCR-robots for analytical and diagnostic applications are available, these can only be operated economically with a constantly high number of samples. In food analytics in particular, there is a demand for analyses for discrete controls or in batches, for example in the case of suspected contamination. Individual or batch analyses are difficult to automate, because for each investigation different primers and sample work-ups are required.

DE 198 40 531 (WO 00/12756; CA 2 342 581) discloses reaction vessels having interior walls coated with predefined amounts of primers and, if necessary, known amounts of a reference nucleic acid, and wherein the interior walls are not otherwise chemically or biochemically modified. In practice, the coating of the interior walls is carried out by “mild lyophilisation” of an aqueous nucleic acid solution in the plastic reaction vessel, such that the nucleic acids are adsorbed onto the interior wall. Since such an adsorption is not necessarily reversible, it is further recommended to admix large quantities of non-specific carrier nucleic acids to the specific nucleic acids. In BioTechniques 18(6): 981-984, Day INM et al. (1995) disclose the storage of a dried matrix-DNA (reference DNA) and of PCR starter oligonucleotides in micro-titre plates in connection with a genetic analysis. In the genetic analysis, the amount of DNA in the sample is known, or the amount of matrix-DNA in the reaction vessel is not relevant. The method described is not suitable for quantitative analyses or the determination of unknown amounts of DNA. However, in many areas of food analytics and diagnostics, the concentration of a specific nucleic acid (DNA or RNA) in the sample has to be precisely determined and one prerequisite for such quantitative investigation is the availability of a precise standard dilution series. At very low concentrations of 1 to 100000 molecules per reaction, even nucleic acids become “unstable”, or the molecules are unavailable to react in the reaction vessel. This is remedied by adding a larger amount of non-specific DNA to the low concentration specific nucleic acid, which has practically no sequence homology with the nucleic acid to be detected. However, this is problematic for many reasons and may lead to many systematic errors, and therefore it is generally recommended to carry out all necessary dilution steps daily, starting from a stock solution with a defined concentration. This is very labour intensive and is subject to varying precision of the pipetting, which reduces the reliability and reproducibility.

International patent application WO 2004/106549 discloses a method for the maintaining of nucleic acids, in which aqueous solutions of the nucleic acid are lyophilised in the presence of disaccharides and collagen, preferably trehalose and collagen. Trehalose is essentially supposed to make the nucleic acids more durable and collagen is supposed to stick to and protect the nucleic acid present on the glass surface.

International patent application WO 2005/103277 teaches a dry amplification mixture for PCR and the technical PCR analysis, comprising DNA-polymerase, deoxyribonucleosides, buffer components, water soluble dyes for the DNA-electrophoresis and stabilisers, D-glucose, disaccharides such as innulin, sucrose, trehalose, and maltose, and polysaccharides such as D-mannitol, dextrans, phycoll, polyvinyl pyrrolidone etc. The PCR-method comprises the dissolution of the dry amplification mixture in a buffer with magnesium ions and the subsequent addition of primer and the DNA-sample to be analysed.

EP-A2-1 374 827 discloses methods for the stabilisation of dry and partially dry mixtures with PCR-enzymes and reagents, as well as kits comprising these dry mixtures. These mixtures are only durable for a short time, even when kept in a cooling chamber.

In Appl. Environ. Microbiol. (2005) 71 (11) 6702-10, Tomlinson J A et al. describe a reagent set for the detection of pathogens in a field trial. They describe, among others, reaction vessels comprising lyophilised real-time PCR-reagents, which are durable at room temperature; the long-term stabilisation of PCR-reagent mixtures by trehalose and a kit with internal PCR-controls for plant DNA, which are supposed to show the success of the DNA-extraction. The vessels comprise enough reagents for 10 PCR-experiments, wherein water has to be added before the experiments.

In J. Clin. Microbiol. (1998) 36 (6) 1799, FIG. 2, Klatser P R et al disclose the long-term stabilisation of PCR reagent mixtures by trehalose.

In IEEE (2006), Proceedings of the 1^{st} Distributed Diagnosis and Home Healthcare (D2H2) Conference, Arlington, Va., USA, Apr. 2-4, 2006, the article Dry-reagent storage for disposable lab-on-a-card diagnosis of enteric pathogens, by Ramachandran S et al describes investigations into the stabilisation of PCR-reagents using trehalose. The reagents are placed on a microfluidic card, wherein trehalose inhibits the formation of primer-dimers during storage (same reference, page 18, right column).

This state of the art poses a problem. In particular, one disadvantage of the state of the art is that despite all the preventive measures, the amounts of PCR active DNA present in the PCR-vessels vary, even in the cases where equal amounts of DNA were introduced into the vessels. This is presumably due to the fact that the PCR reagents—deoxy(ribo)nucleotide triphosphates, primer oligonucleotides and matrix RNA or DNA—are dried by lyophilisation in the vessels either separately or in a mixture together with the special substances or stabilisers (unspecific DNA, gelatine, collagen, glucose, innulin, maltose, mannitol, dextrans, trehalose, sucrose, and other disaccharides, THESIT®, polyethylene glycol, polyvinyl pyrrolidone, TRITON-X 100®, TWEEN-20®, bovine serum albumin (BSA), phycoll, buffer salts, such as Tris-HCl, KCl, DTT, etc.). Such dried PCR-mixtures are highly hygroscopic and lose activity and efficiency during longer storage, due to take-up of moisture. This effect is further amplified by the addition of stabilisers such as collagen and gelatine. If lyophilisation is carried out in the absence of paste-forming substances, such as collagen or gelatine, the PCR-reagents form flakes, which may migrate in the vessels, such that no quantitative analysis may be possible in the prepared vessels.

There is therefore a need for a PCR reagent kit, which is in particular suitable for sporadic samples, which does not require particular tools and may be used routinely in any laboratory immediately and without special preparations for quantitative and analytical investigations by a qualified person, such as a chemical-technical assistant. It is further an object of the invention to solve the problems of the state of the art.

### BRIEF DESCRIPTION OF THE INVENTION

The problem is solved by a packaging unit with prepared reaction vessels according to claim 1 and the inventive method according to claim 15. Preferred embodiments of the invention may be derived from the dependent claims.

The inventive packaging unit for carrying out PCR for analytical, diagnostic and technical purposes comprises a set of labelled dry reaction vessels in storage form, with a series of known amounts of primer-oligonucleotides, which start an amplification of the target sequence in a PCR after addition of suitable reagents, enzyme and DNA or RNA sample; a set of labelled dry reaction vessels in storage form with a series of known amounts of primer-oligonucleotides and reference nucleic acid, which, after addition of predetermined amount of liquid, give a concentration series of the reference nucleic acid, wherein the reference nucleic acid comprises the target sequence. According to the invention, both sets of labelled dry reaction vessels in storage form are prepared such that aqueous solutions of known amounts of primer-oligonucleotides with and without reference nucleic acid are dried in the reaction vessels at a temperature of 5 to a maximum of 30° C. above room temperature under ambient pressure, solely in the presence of 1 to 5 mMol/L trehalose, such that the resulting pellet is completely dry, but not hygroscopic. In an alternative embodiment of the invention, both sets of labelled dry reaction vessels are produced such that in the reaction vessels aqueous solutions of known amounts of primer-oligonucleotides with and without reference nucleic acid are gently dried at ambient temperature under a reduced pressure of 0.1 to 0.3 bar, solely in the presence of 1 to 5 mMol/L trehalose, in such a way that the resulting pellet is completely dry, but not hygroscopic, and that the trehalose does not crystallise.

The state of the art consistently teaches lyophilisation of the oligonucleotide-primer or the reference nucleic acid, in order to ensure their stability. However, lyophilisation of nucleic acids or oligonucleotides often leads to the appearance of lints, such that no storable reaction vessels may be obtained. Also, the “active drying” or lyophilisation of buffers or amplification mixtures often leads to surface reactions. Close hydrogen bonds may be formed with hydroxyl- and other oxygen-containing groups on the wall of the reaction vessels, whereby even nucleic acids become partially insoluble. It then becomes necessary to separate the nucleic acids and oligonucleotides from the surfaces in a separate step. Therefore, in the state of the art, hydroxyl containing hydrogen bond forming molecules, such as unspecific DNA, gelatine, collagen, glucose, innulin, maltose, mannitol, dextrans, trehalose, sucrose, and other disaccharides, THESIT®, polyethylene glycol, polyvinyl pyrrolidone, TRITON-X 100®, TWEEN-20®, bovine serum albumin (BSA), phycoll, and others are added to the dry mixtures, in order to “stabilise” the oligonucleotides and nucleic acids in the intended very low concentrations, and in order to keep the pellet on the vessel wall. Since such buffer solutions only dry slowly and unsatisfactorily, these solutions are normally lyophilised, which leads to hygroscopic pellets, in which the nucleic acids and the primer-oligonucleotides are not stable. The inventors have discovered that drying at slightly elevated temperature in the presence of only a low concentration of trehalose leads to much better results. However, too much trehalose or other buffer salts leads to flake formation during the drying. According to the invention, a glass-hard trehalose layer is formed during drying under heightened temperature over 1 to 4 hours, which strongly adheres to the surface of the vessel wall, and in which the primer-oligonucleotides and/or the reference nucleic acids are embedded. Only during such a drying process is the trehalose able to displace the water molecules in the hydrogen bonds, such that the nucleic acids and oligonucleotides remain stable in the required, very low amounts.

A further aspect of the invention concerns the combination of uniformly produced reaction vessels with primer-oligonucleotides or nucleic acids. It is surprising that even a small copy number of the target sequence, such as 100 to 1000 is stable, if the aqueous solution is dried only in the presence of trehalose. Because the reaction vessels with the primers and the reference nucleic acid are produced in the same way, absolute comparability between the sample and the reference is ensured.

One preferred embodiment of the invention concerns a packaging unit with the mentioned reaction vessels, wherein the volume of the aqueous solutions in the reaction vessels prior to drying is 1 to 25 μL. The concentration of primer is between 0.1 and 100 μMol/L with 1 to 5 mMol/L trehalose. The target sequence is preferably present in the reaction vessels with the reference nucleic acid in an amount of 10 to 100 000 units, as genomic DNA or as plasmid. An amplificate may also be used. It was found however, that amplificates are much less stable under these conditions. Presumably, a nuclease exo-activity is introduced when amplificates are used. The copy number per reaction vessel is preferably between 100 and 5000.

A further embodiment concerns reaction vessels for a Hot-Start-PCR. In this case, the dried primers or reference nucleic acid are covered by a hydrocarbon wax, which melts at 57° C. and floats to the top of the aqueous solution. This prevents primers and nucleic acids from already hybridising with each other at low temperatures. The same effect may also be achieved by using hot-start polymerases, which only become active at temperatures above 50° C., although Hot-Start polymerases are considerably more expensive than conventional polymerases and reverse transcriptases. The layer of high melting temperature hydrocarbon wax also protects the underlying pellet with the primers and/or reference nucleic acid from moisture.

In an especially preferred embodiment of the invention, the kit comprises reaction vessels, in which spatially separated primer-oligonucleotides and reference nucleic acid are dried onto the vessel wall in the presence of trehalose, in such a way that, after addition of the aqueous solution with the further reagents for the PCR, primer-oligonucleotides and reference nucleic acid are dissolved in the aqueous solution.

For practical reasons, the vessels are preferably formed as wells in a microtiter plate. Microtiter plates are normally commercially available as plates with 24, 48, 96, 192 or 384 wells.

The amount of the two primers in the reaction vessels is preferably set to 7.5 pmol (2.5 to 15 pmol), and the amount of reference nucleic acid to 100 to 1000 copies of the target sequence. Namely, in the first phase of the amplification, the amount of matrix (target sequence) is limited, and the probability that matrix, primer and polymerase meet is suboptimal, while, during the third phase of the amplification, the amount of products (DNA, pyrophosphate, monophosphate nucleotides) increases to such an extent that they inhibit the reaction, that more product fragments hybridise with each other, and that the substrates are slowly used up and finally the polymerases and nucleotides are slowly destroyed by the heat. An exponential increase, which is quantifiable when using fluorescence for real-time PCR, only occurs during the phase in between. The PCR remains exponential for about 30 cycles in case of 12 to 400 starting copies, for 25 cycles in the case of 200 to 3200 starting copies, and for maximum 20 cycles in the case of 3000 to 50 000 starting copies. In order to be able to measure at the beginning of the exponential phase, the CT-value (threshold cycle) or the Cp-value (crossing point) is often used, which describes the cycle during which the fluorescence significantly rises above the background fluorescence for the first time.

The packaging unit may further comprise one or more of the following buffer and reaction solutions, for example DNA or RNA-extraction solution, proteinase-K solution, gel-loading buffer, nucleotide and amplification buffer (MasterMix), DNA-polymerase or reverse transcriptase. Since highly perfected amplification mixtures, optimised for all possible means, are commercially available in ready-to-use form, such as for example AmpliTaqGold® MasterMix of Roche Molecular Systems, Inc., users will still trust the mixtures used thus far, such that the last named option—including MasterMix—is only mentioned for completeness.

According to the invention, only primer-oligonucleotides and reference nucleic acid are present on the interior wall of the reaction space or the reaction vessel, embedded in a trehalose-film. Trehalose (also known as mycose) is a non-reducing disaccharide, which is formed from two α-1,1-glycosidally linked D-glucose molecules, which can form hydrogen bonds with proteins and nucleotides; see Colaco C et al (1992) in Bio/Technology 10, 1007-1011. Trehalose is therefore a strong PCR-enhancer, which on one hand reduces the melting temperature in solution and on the other hand thermally stabilises the Taq-DNA-polymerase (Spiess A N at al (2004) Clinical Chemistry 50(7), 1256-1259). It is further taught, in a Hot-Start PCR, to dry part of the reaction components in the presence of trehalose and to embed them in a wax, which melts at about 57° C. (see Kaijalainen et al. (1993) Nucleic Acids Res., 21(12):2959-2960). The reaction components, which are embedded in the wax droplet, are then only released to the other reaction components of the PCR-assay upon the melting of the wax-coating at higher temperatures, which may avoid a mispriming and an early start of the DNA-polymerase reaction at low temperatures. However, this process is only suitable for very high sample numbers, since the embedding of part of the reaction components in a wax droplet on a polyethylene wire is complicated. There is no teaching of long-term durability of primers and reference nucleic acid on the polyethylene wire. According to the present invention, in contrast to the wax droplet technique, the primers and/or reference nucleic acid are dried onto the wall of the reaction vessel in the presence of trehalose, preferably on the base of a vessel. Such specifically prepared sample vessels may then be activated by addition of a defined amount of water and amplification mixture (DNA-polymerase, dNTPs, Mg²⁺, Tris-HCl buffer, pH 8.0). Further steps are not necessary, since the labelled sample vessels already comprise the primers and a predetermined number of copies of the target sequence in known amounts, protected by a glass-like layer of trehalose, which completely dissolves under the conditions of a PCR. The trehalose probably acts as a dissolution aid for the few copies of reference nucleic acid.

While it is known to pre-introduce the necessary reagents for an enzymatic reaction into a reaction vessel, and to dry, if required, the reagents onto the surface of the sample vessel, it is problematic that the reagents crystallise differently from each other during the lyophilisation or drying, and form small flakes, which do not adhere to the surface and may disperse throughout the whole vessel. If one wants to concentrate all of the reagents as a pellet at the base of the vessel, large amounts of salts are required. This may interfere with the subsequent enzymatic reaction, and they may also bind water through which the stability of the different reagents may no longer be ensured. According to the invention, this problem is solved by the elimination of superfluous reagents, and by drying the oligonucleotides and the reference nucleic acids only in the presence of physiological amounts of trehalose onto an inert substrate, such as a polyethylene wall. Not all sugars are suitable, only those that form a glass-like layer upon drying. At the same time, the sugar has to dissolve quickly in the presence of water.

These requirements are ideally met by trehalose. Trehalose is weakly hygroscopic and has a comparably high gelation and glass transition temperature. In nature, trehalose protects cells from injuries from ice crystals during frost or deep-freeze conditions and also during drought periods. In a certain way, trehalose is functionally equivalent to saccharose, but has different glass-point and stabilising properties. Trehalose is naturally present in plants and fungi, and in the hemolymph of many insects. Trehalose is chemically and thermally stable, stable to acid and quickly soluble in water, whereby trehalose is less soluble than saccharose at low temperatures and more soluble at high temperatures. As opposed to the disaccharides, trehalose is not hydrolysable and does not take part in a Maillard reaction with amino acids or proteins. It also has a high adherence on plastic walls.

Therefore, reaction vessels for PCR are available, which may be stored for months and years at room temperature. When required, the reaction vessels may be used immediately. Hence even small and medium-sized laboratories with discontinuous or sporadic sample requirements of quantitative PCR analyses may manually process these immediately and without large effort. All primers and reference nucleic acids are available in exact amounts and for immediate use in the reaction vessels, and only a sample DNA or RNA prepared for the specific analysis reaction and a Mastermix (amplification mixture) need to be added. However, this is always necessary. According to the invention, possible errors caused by a wrong addition or a wrong determination of amounts of primer and reference nucleic acids are eliminated. In a preferred embodiment, vessels with the “wrong” nucleic acid or further positive and negative controls are supplied as well as the inventive sample vessels.

Ready-to-use master or amplification mixtures may be commercially obtained from a multitude of suppliers, with varying natural or genetically modified or chemically modified DNA polymerases or reverse transcriptases. Typically employed DNA-polymerases are Taq-DNA-polymerase, the recombinant truncated form of Taq-DNA-polymerase, which lacks the 5′-3′-exoactivity (KlenTaq), a chemically modified Taq-DNA-polymerase for a Hot-Start PCR, or a high-fidelity recombinant thermostable DNA-polymerase from Pyrococcus abyssii, etc. The master mixtures may be repeatedly concentrated, and may or may not comprise magnesium ions. This means that the magnesium ions may be added from a separate magnesium stock (for example 25 mM MgCl₂). The Mastermixes may further comprise compounds which increase the sample density, such that the products from the PCR may be added directly to the pockets of an analytical or quantitative agarose gel. The Mastermix may further comprise dyes, either for a real-time PCR or for analysis on the agarose gel.

### DESCRIPTION OF THE FIGURES

Further problems, advantages and solutions of the inventions may be derived from the subsequent examples and the figures:

FIG. 1 shows a gel electrophoresis of the amplification products in labelled reaction vessels with different samples and reference nucleic acids according to the invention

FIG. 2 shows a gel electrophoresis of the amplification products in labelled reaction vessels with conventional samples and dry samples of the reference nucleic acid according to the invention in an ageing experiment of 6 months at 37° C. (comparison of the ageing behaviour);

FIG. 3 shows a gel electrophoresis of the amplification products in reaction vessels according to the invention with different amounts of added hazelnut DNA and ageing over 6 months at 37° C. (sensitivity experiment).

### DETAILED DESCRIPTION OF EMBODIMENTS OF THE INVENTION

### Examples

### Example 1

### Test for the Detection of Hazelnut (Corylus Avellana) in Food Products

Labelled polypropylene reaction vessels were supplied, in the wells of which (200 μL) known amounts of specific primers for the detection of hazelnut and whole hazelnut DNA as the reference nucleic acid were dried. This corresponded to a mixture of 0.75 μL first primer CaMAV-F3 with a concentration of 10 μMol/L, 0.75 μL second primer Ca-R05 with a concentration of 10 μMol/L, 2 μL of a 5 mMol/L trehalose solution and 0.5 μL water. The reference nucleic acid was 200 pg genomic hazelnut DNA, corresponding to 100 copies of the target sequence (amplicon sequence), and the five-fold and ten-fold thereof, respectively dissolved in 0.5 μL and added to the solution instead of water. The drying time was 3 hours at 40° C. under ambient pressure and was carried out in a dry heating oven. The reaction vessels were closed until use. No further PCR probes for real-time PCR were dried, even though that would be possible at this stage. At the end, the prepared wells of the microtiter plates contained the primers required in the optimal amount for the determination of hazelnut DNA in the sample or for the negative control (checking for contamination of the master mixture) and for the extraction control (checking of the extraction for contamination). The reaction wells labelled in red contained low amounts of hazelnut DNA in addition to the primers. They allow a positive control on one hand (functional capability of the master mixture), an inhibition control on the other hand (checking of the DNA-isolate for inhibitors) and further a quantification of the DNA in the sample.

DNA-extraction was carried out according to the CTAB-procedure (ISO 21571, Foodstuffs—Methods of analysis for the detection of genetically modified organisms and derived products—Methods for nucleic acid extraction) with subsequent purification on a silica matrix. The samples were homogenised, weighed into 15 mL centrifuge tubes in 1 g portions, 10 mL CTAB extraction solution (2% cetyl trimethyl ammonium bromide, 1.4 mol/L NaCl, 0.02 M EDTA, 0.1 mol/L Tris-HCl, pH 8.0) and 50 μL Proteinase-K (10 mg/mL) were added, mixed and incubated under shaking for at least 90 minutes at 60° C. 1 mL liquid was removed from each sample, centrifuged at 17000×g for 5 minutes, and then 750 μL supernatant was extracted with 300 μL chloroform/isoamyl alcohol “Ready Red™” (MB Biomedicals, Illkirch, France). This was followed by a further centrifugation at 17000×g for 5 minutes. 500 μL aqueous supernatant was then treated with 300 μL isopropanol, mixed, and the precipitating DNA was pelletised for 10 minutes at 17000×g. The pellet was washed with 500 μL ethanol (70%), and again pelletised for 5 minutes at 17000×g. The supernatant was removed, the DNA-pellet briefly dried on air and then dissolved in 100 μL water (if necessary using ultrasonic treatment).

Further DNA-purification was carried out using QIAquick® PCR purification kit of Qiagen GmbH, Hilden, Germany. 500 μL PB-buffer (Qiagen GmbH) was added to 100 μL DNA extract, well mixed, the solution loaded onto the column, and the column was centrifuged for 1 minute at 17000×g. The flow-through was disposed of, 750 μL PE-buffer (Qiagen GmbH) was loaded onto the column and centrifuged for 30 seconds at 17000×g. The flow-through was disposed of again and the column centrifuged for another 30 seconds at 17000×g without further loading. The collecting tube was disposed of and the column introduced into a new reaction vessel. After loading of the column with 250 μL EB-buffer, it was centrifuged for 1 minute at 17000×g, the column was disposed of and the eluate used for the PCR. All the procedures were carried out using the usual protective measures. For the avoidance of carryovers, it is recommended to use protective clothing as well as micropipettes with filter tips.

The isolated and purified DNA was amplified in the reaction wells of the microtiter plate. 12.5 μL DNA extraction and 12.5 μL 2× AmpliTaqGold® Mastermix of Applied Biosystems were added to the wells. An amplification of the target sequence on a thermocycler (Eppendorf Mastercycler) followed. In this case, the cycler profile was 10 minutes at 95° C. as initial activation of the polymerase, 15 seconds at 95° C. and 60 seconds at 62° C. for 45 cycles. If necessary, the time profile has to be adapted. 5 μL amplificate (78-base-pair amplificate) was then analysed after addition of loading buffer on a 2.5%-agarose gel (2 to 4 μL ethidium bromide in TAE-buffer) and usual electrophoresis (10 minutes at 3 to 6 V/cm), and the product was visualised on a transilluminator.

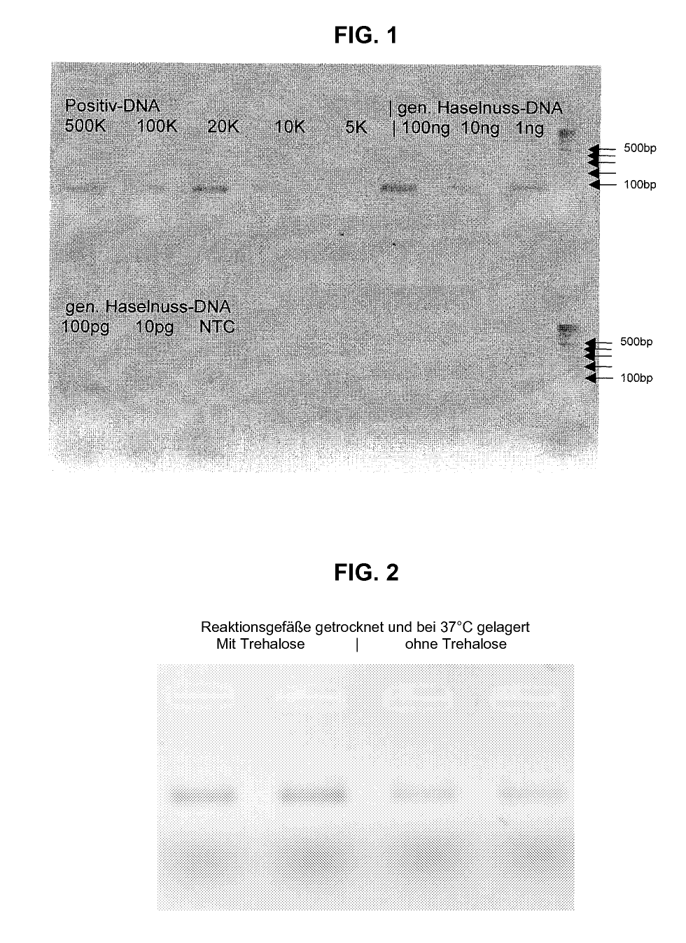

In order to obtain a correct result of the analysis, a positive reference with the target sequence had to show a band of length 78 base pairs, and the negative control was not to show a band in this area (see FIG. 1). If the sample showed a band at the same height than the reference, the reaction was validated as a positive. If there was no band, this could only mean that no hazelnut DNA was present in the sample, or that the reaction had been inhibited. Inhibition was ruled out if the same DNA-isolate in a well with reference nucleic acid was clearly positive. If that was not the case, there was inhibition of the reaction and the DNA-isolate was amplified at a higher dilution.

All sample combinations may be tested on the inventive microtiter plate with the standardised amounts of reference and control nucleic acid and the optimised primer concentrations, since the case that the reference nucleic acid would be present in insufficient amounts due to decomposition does not occur.

The sequence identity of the amplificate may then additionally be tested by restriction, for example using BamH I. In the case of hazelnut DNA, this leads to two fragments with respective lengths of 20 pb and 58 pb. The detection limit for genomic hazelnut DNA is approximately 50 pg. The present reaction was specific against 100 ng DNA of any of the following species: peanut, almond, cashew nut, macadamia nut, walnut, pecan nut, pistachio, apricot, corn, soybean, celery, brassica, orange, mandarin, brazil nut, wheat, rye, barley, oat, spelt, fagopyrum (see FIG. 1).

By intensity comparison with the reference nucleic acid, a quantification of the positive sample DNA is also possible.

### Example 2

### Ageing and Relative Sensitivity at Drying in the Presence and Absence of Trehalose; Stress Tests for the Assessment of the Storability/Durability/Yield

The labelled reaction vessels were produced as described in Example 1, with the difference that a number of reaction vessels with reference DNA were dried in the presence of trehalose and a number of reaction vessels with reference DNA were dried in the absence of trehalose. The reaction vessels were then stored for 6 months at 37° C. For the amplification, the reaction vessels were filled with 12.5 μL two-fold concentrated MasterMix (AmpliTaqGold® MasterMix) and 12.5 μL water, closed and put into the PCR-cycler (Eppendorf Mastercycler). The cycler profile was identical to the profile in Example 1. The subsequent gel electrophoresis showed a band at 78 bp for both reaction vessels. However, the intensity of the bands from the reaction vessels without trehalose was only about ⅓ of the band intensity from the reaction vessels with trehalose (see FIG. 2).

This shows that the reaction vessels in which the primer and reference DNA were not dried in the presence of trehalose give a clearly lower yield in the amplification, and hence have lower sensitivity, if they have been stored over a longer period.

### Example 3

### Sensitivity after Storing at 37° C.

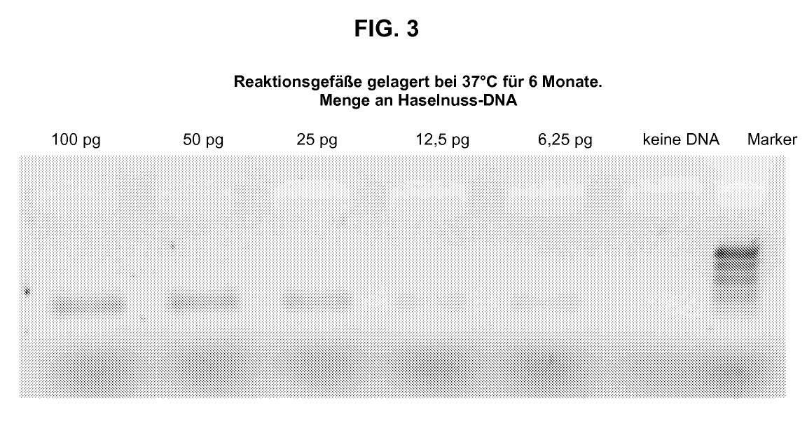

The labelled reaction vessels were produced as described in Example 1 and stored for 6 months at 37° C. For the amplification, the reaction vessels were filled with 12.5 μL two-fold concentrated MasterMix (AmpliTaqGold® MasterMix) and 12.5 μL DNA-isolate, closed and put into the PCR-cycler (Eppendorf Mastercycler). The DNA-isolates comprised hazelnut DNA in amounts 100 pg, 50 pg, 25 pg, 6.25 pg and no hazelnut DNA. The cycler profile again was identical to that of Example 1. The gel electrophoresis showed amplification of a 78 bp long fragment in all the reaction vessels, except in the one which did not contain any hazelnut DNA (see FIG. 3).

Hence, if the primers are dried in the presence of 5 mMol/L trehalose, they maintain their activity, also after longer storage at elevated temperature.

### Example 4

### Application in Real-Time PCR

To each reaction vessel was added a mixture of 0.45 μL first primer CaMAV-F3 with a concentration of 50 μMol/L, 0.45 μL second primer Ca-R05 with a concentration of 50 μMol/L, 0.625 μL probe CaMAV-S1 with a concentration of 10 μMol/L, 2 μL trehalose solution of 5 mMol/L and 0.475 μL water. The reference nucleic acid was 200 pg genomic hazelnut DNA, corresponding to 100 copies of the target sequence (amplicon sequence), dissolved in 0.475 μL, which was added to the solution instead of water. The drying was carried out over 3 hours at 40° C. under ambient pressure in a dry heating oven. The reaction vessels were then wrapped in foil and stored in the dark until use.

For the amplification, 12.5 μL Universal MasterMix® of Applied Biosystems and 12.5 μL DNA-isolate were added to each reaction vessel and the reaction vessels were put into a real-time PCR machine (SDS 7500 of Applied Biosystems). Amplification was carried out according to the same temperature profile as in Example 1.

Hazelnut-DNA-isolate was added as a matrix in different concentrations. The number of copies per reaction vessel was between 20 and 100 000. Each number of copies was amplified in triplicates. The results are shown in Table I:

TABLE 1
Real-time PCR in reaction vessels prepared according to the invention
20K-100 000K

| Copies | Ct MW | Gradient |
|---|---|---|
| 100 000 | 24.77 | −3.10 |
| 10 000 | 28.88 | Axis |
| 1000 | 32.05 | 40.82 |
| 100 | 34.48 | Correlation |
| 40 | 35.93 | 0.991 |
| 20 | 36.42 | |

The obtained Ct-values show a good linearity over the whole range of copy numbers.

### Example 5

### Application in Fluorescence-Endpoint Determination

The reaction vessels according to the invention may be standardised such that they allow PCR-analysis using endpoint determination. This is described here for the determination of salmonella DNA:

25 g (mL) of a food sample were weighed into a sterile Stomacher bag, diluted 1:10 (w/v) with 225 mL buffered peptone water and pre-incubated for 18 h at 37° C. Selective reproduction of the salmonella followed. To this end, 0.1 mL pre-incubate was transferred into a culture tube with 10 mL RVS-bouillon and incubated for 5 to 6 hours at 42° C.

For the DNA-extraction, 1 mL of the selective culture was centrifuged for 5 minutes at 10000×g, the supernatant removed, the pellet re-suspended in 0.2 mL 0.1×TE-buffer and heated to 95° C. for 10 minutes. After cooling, the probe was centrifuged for 5 minutes at 14000×g, and the supernatant containing the DNA was directly used in the PCR.

For the PCR with SYBR-Green fluorescence-endpoint determination, the cavities of a microtiter plate were filled with pre-dried primers or reference-DNA (PCRFast salmonella, Lot TSAL_{—}39531) and 12.5 μL MasterMix (Power SYBR® Green PCR MasterMix, Applied Biosystems Nr. 4367659). Then, 12.5 μL of the DNA-isolates from the food samples and the dilutions were added, the strips closed and put in the PCR-thermocycler (STRATAGENE Mx 3005 P). Amplification was carried out using the temperature profile: 10 minutes at 95° C., followed by 30 cycles with 15 seconds at 95° C. and 60 seconds at 67° C.

Measurement of the fluorescence after the last cycle (R Last/fluorescence-endpoint determination, stimulation by white light from a halogen lamp and emission measurement at 520 nm, no unit) at the end of the amplification gave the following values:

TABLE 2

| | R Last |
|---|---|
| Positive reactions with target-DNA | |
| Sample 1 | 24693 |
| Sample 2 | 24513 |
| Positive control | 22074 |
| Reactions without target-DNA | |
| Sample 3 | 6439 |
| Sample 4 | 7091 |
| Negative control | 5444 |

PCR-conditions (probe detection): wells with pre-dried primers and sample (PCRFast salmonella, Lot RSAL_{—}39239) were treated with 12.5 μL MasterMix (AmpliTaq Gold® PCR MasterMix, Applied Biosystems Nr. 4318739). Then, 12.5 μL of the DNA-isolates from the food samples were added, the strips closed and put in the PCR-thermocycler (STRATAGENE Mx 3005 P). Amplification was carried out using the following temperature profile: 10 minutes at 95° C., followed by 35 cycles with 30 seconds at 95° C., 45 seconds at 60° C., and 30 seconds at 72° C. Measurement of the fluorescence values (R Last, stimulation by white light from a halogen lamp and emission measurement at 520 nm, no unit) at the end of the amplification gave the following fluorescence values for the endpoint determination.

TABLE 3

| | R Last |
|---|---|
| Reactions comprising target-DNA | |
| Sample 1 (A1) | 51728 |
| Sample 2 (A2) | 52071 |
| Positive control (B8) | 51374 |
| Reactions without target-DNA | |
| Sample 3 (A7) | 29133 |
| Sample 4 (B5) | 26672 |
| Negative control (B6) | 29590 |

Both experiments show that the measurement difference in the fluorescence-endpoint determination between the positive and the negative control, or the samples, is so large each time that one could easily differentiate between the presence and absence of salmonella in the food sample without complex measurement. For the measurement, stimulation using white light from a halogen lamp and emission measurement at 520 nm (without unit) was sufficient. Hence, salmonella-testing may be carried out using PCR in very basically equipped laboratories. Preferably, reaction vessels are used that are transparent at the relevant wave lengths, preferably made of polycarbonate.

### SUMMARY

With the reaction vessels prepared according to the invention, biomolecular food analyses may be carried out in a simple and standardised manner. Hence, innovative products are available for the simple biomolecular determination of specific DNA-fragments in foodstuff and fodders. The prepared reaction vessels may be adapted to all relevant parameters in the areas of allergens, GMOs, animal species and hygiene.

This is because the most important key reagents are provided in the inventive PCR-vessels ready-to-use in optimal amounts: specific primer sequences and the necessary specific positive controls. Hence, all the laborious pipetting steps and measures for ensuring the quality and avoiding possible contamination during the PCR-process are omitted. The species-specific DNA-controls allow the flawless checking of the PCR, such as the provision of a positive control (checking for the functional capability of the Mastermix) and the checking for inhibitory effects (inhibition control) in the extracted sample-DNA. At the same time, the testing kit allows the negative control (water control) and the checking of a contamination-free extraction (extraction control). These controls are equivalent to the analyte and allow a practical inhibition control of the matrix.

Only the DNA of the sample has to be isolated according to a standardised sample work-up protocol. DNA work-up kits as suggested and validated by the prior art are available and may be added to the packaging unit. After extraction of the DNA from the sample material, only two pipetting steps are required for the work-up of the PCR: 12.5 μL of sample-DNA and 12.5 μL two-fold concentrated MasterMix, comprising polymerase, nucleotides, magnesium chloride and buffer in optimum concentrations are pipetted into the reaction vessel. The user takes the required number of reaction vessels from the test kit and can simply put the non-required reaction vessels back into storage. The reaction vessels are preferably clearly arranged in a rack, analogously to a microtiter plate.

The test or the reaction vessels with the reagents may be stored for up to 2 years at 2 to 10° C. Apart from the primers, the labelled reaction vessels also comprise the control-DNA. The MasterMix may be obtained through leading suppliers, such as Applied Biosystems Inc. The Universal MasterMix is adapted to all parameters, meaning that only one MasterMix is required for the complete product line. This considerably reduces the work-load in the laboratory and increases safety and reproducibility of the PCR.

The temperature and cycler profile is nearly identical for all parameters and predetermined in the test kit description. This allows simultaneous analysis and determination of different parameters in one run. The user is in a position to assemble a macrochip himself (for example for simultaneous screening of the allergens soybean, hazelnut and peanut, or several GMO parameters in parallel).

Detection is normally carried out in agarose gel with ethidium bromide. Selected parameters may also be considered in real time for quenching in a block cycler.

The following parameters may inter alia be considered for the inventive test kit: foodstuff allergens: hazelnut, almond, walnut, pecan nut, brazil nut, cashew nut, pistachio, peanut, wheat/barley/rye, wheat, celery, mustard, sesame, soybean, fish, lupins. Animal species: pig, cattle, ruminants, mammals, chicken, turkey, duck, poultry, sheep, goat, horse, rodents, dog, cat. GMO: Screen 35S, Screen nos, Roundup Ready Soy (RRS), Cauliflower mosaic virus (CMV), corn MON810, corn MON863, corn BT176, corn BT11, corn GA21, corn NK 603, corn T25, rice LL601, rice LL62. Hygiene: salmonella spp., listeria monocytogenes, campilobacter (jejuni, coli, lari), EHEC, Staphylococcus aureus, Bacillus cereus, Yersinia enterocolitica, Clostridium perfringens, Shigella flexneri.

The advantages of the inventive reaction vessels are:
the test systems are storable
primers and species specific positive controls are supplied ready-to-use
no liquid handling of primers and controls required
no more risk of contamination
simple extension to new parameters and products
total standardisation of the PCR
independent of sample work-up
several parameters may be tested simultaneously in one run
only a single Universal MasterMix required for all parameters

Biomolecular food analyses play an increasingly important role in quality control. Specific DNA fragments may be detected and visualised by PCR with the method. These days, unsolved analytical problems are clearly resolvable with the DNA-analysis. As recent examples, the detection of apricot seeds in marzipan or specific detection of allergy-causing substances such as mustard or celery may be cited.

For the identification of genetically modified organisms, PCR has an equally important role as in the pathogenic-hygienic area. Hence pathogenic germs may be detected early and quickly, and storage times until analytical release may be considerably reduced. With the inventive reaction vessels, the standardisation of PCR in all areas of food industry and analytical laboratories becomes possible.

1. Packaging unit or kit for carrying out PCR for analytical, diagnostic and technical purposes, comprising
a set of labelled dry reaction vessels in storage form, with a series of known amounts of primer-oligonucleotides, which start an amplification of the target sequence in a PCR after addition of suitable reagents, enzyme and DNA or RNA sample;
a set of labelled dry reaction vessels in storage form with a series of known amounts of primer-oligonucleotides and reference nucleic acid, which, after addition of predetermined amounts of liquid, give a concentration series of the reference nucleic acid, wherein the reference nucleic acid comprises the target sequence,
characterised in that both sets of labelled dry reaction vessels in storage form are prepared such that aqueous solutions of known amounts of primer oligonucleotides and/or reference nucleic acid are dried in the reaction vessels at a temperature of 5 to a maximum of 30° C. above room temperature under ambient pressure solely in the presence of 1 to 5 mMol/L trehalose, in such a way that the resulting pellet is completely dry, but not hygroscopic.

2. Packaging unit or kit for carrying out PCR for analytical, diagnostic and technical purposes, comprising
a set of labelled dry reaction vessels in storage form, with a series of known amounts of primer-oligonucleotides, which start an amplification of the target sequence in a PCR after addition of suitable reagents, enzyme and DNA or RNA sample;
a set of labelled dry reaction vessels in storage form with a series of known amounts of primer-oligonucleotides and reference nucleic acid, which after addition of predetermined amounts of liquid give a concentration series of the reference nucleic acid, wherein the reference nucleic acid comprises the target sequence,
characterised in that both sets of labelled dry reaction vessels are produced such that in the reaction vessels aqueous solutions of known amounts of primer oligonucleotides and/or reference nucleic acid are gently dried at ambient temperature under a reduced pressure of 0.1 to 0.3 bar solely in the presence of 1 to 5 mMol/L trehalose, in such a way that the resulting pellet is completely dry, but not hygroscopic.

3. Kit according to claim 1, wherein the volume of the aqueous solutions in the reaction vessels before drying is 1 to 25 μL and the amounts of primer between 0.1 and 20 nMol/L.

4. Kit according to claim 1, wherein the reaction vessels with the reference nucleic acid comprise the target sequence in an amount of 10 to 10,000 copies,
preferably as genomic DNA, or plasmid.

5. Kit according to claim 1, wherein the dry pellet on the vessel wall is covered by a hydrocarbon wax, which starts to melt at a temperature of 57° C. and floats upwards in an aqueous solution.

6. Kit according to claim 1, also comprising reaction vessels in which spatially separated primer-oligonucleotides and reference nucleic acids are dried onto the vessel wall in the presence of trehalose in such a way that, after addition of the aqueous solution with the additional reagents of the PCR, primer-oligonucleotides and reference nucleic acid are dissolved in the aqueous solution.

7. Kit according to claim 1, wherein the reaction vessels are wells of a microtiter plate.

8. Kit according to claim 1, wherein the amount of both primers in the reaction vessels is standardised at 7.5 to 15 pMol, and the amount of reference nucleic acid at 100 or 1000 copies of the target sequence.

9. Kit according to claim 1, further comprising one or more of the following buffer and reaction solutions: DNA or RNA extraction solution, proteinase-K solution, gel loading buffer, nucleotide and amplification buffer, DNA-polymerase, AmpliTaqGold MasterMix®.

10. Kit according to claim 1, wherein the reaction vessels comprise FRET-probes labelled for a real-time PCR, such as hybridisation probes (LightCycler® probes), hydrolysis probes (TaqMan© probes), molecular beacons, scorpion-primer or Lux®-Primer.

11. Kit according to claim 1, which is prepared for carrying out a PCR in the presence of an intercalating fluorescent dye, which forms a DNA-fluorescent dye complex with the formed double-stranded DNA, which is quantifiable by specific fluorescence emission.

12. Kit according to claim 11, wherein the fluorescent dyes are selected from the group of SYBR-Green®, SYBR Gold, SYBR Safe, YO (Oxazole Yellow), TO (Thiazole Orange) and PicoGreen®.

13. Kit according to claim 11, wherein the amounts of primer-oligonucleotides and reference nucleic acid with the target sequence in the set of labelled dry reaction vessels in storage form are selected in such a way that, after a predetermined number of PCR-cycles in the presence of the intercalating fluorescent dye a visual or easily detectable target value for the specific fluorescence emission of the DNA-fluorescent dye complex is obtained.

14. Kit according to claim 10, wherein the reaction vessels are transparent to the absorption and emission frequency of the intercalating fluorescent dye.

15. Process for carrying out a PCR for analytical, diagnostic and technical purposes, comprising the use of a set of labelled dry reaction vessels in storage form with a series of known amounts of primer-oligonucleotides, which start an amplification of the target sequence in a PCR after addition of suitable reagents, enzyme and DNA or RNA sample; a set of labelled dry reaction vessels in storage form with a series of known amounts of primer-oligonucleotides and reference nucleic acid, which, after addition of predetermined amounts of liquid, give a concentration series of the reference nucleic acid, wherein both sets of labelled dry reaction vessels in storage form are prepared such that aqueous solutions of known amounts of primer-oligonucleotides with and without reference nucleic acid are dried in the reaction vessels at a temperature of 5 to a maximum of 30° C. above room temperature under ambient pressure solely in the presence of 1 to 35 mMol/L trehalose, such that the resulting pellet is completely dry, but not hygroscopic.

16. Process according to claim 15, wherein the amounts of primer-oligonucleotides and reference nucleic acid comprising the target sequence in the set of labelled dry reaction vessels in storage form with known amounts of primer-oligonucleotides and reference nucleic acid are selected in such a way that, in the presence of a known amount of intercalating fluorescent dye and a predetermined number of PCR-cycles, a visual or easily detectable target value for the specific fluorescence emission of the DNA-fluorescent dye complex is obtained, and, after carrying out of a predetermined number of PCR-cycles, determination of whether the specific fluorescence emission of the DNA-fluorescent dye complex in the reaction vessel with the sample and in the reaction vessel with the reference nucleic acid reaches the target value.

17. Process according to claim 16, wherein the determination of the specific fluorescence emission of the DNA fluorescent dye complex is carried out using a mobile fluorescence measurement device.
